(19) United States
(12) Patent Application Publication
(10) Pub. No.: US 2009/0308393 A1
(43) Pub. Date: Dec. 17, 2009

(54) Medical diagnostic cart and method of use

(76) Inventor: Wilfredo P. Luceros, Long Beach, CA (US)

Correspondence Address: PATENT LAW & VENTURE GROUP, 2424 S.E. BRISTOL, SUITE 300, NEWPORT BEACH, CA, 92660, US

(21) Appl. No.: 12/214,117

(22) Filed: Jun. 17, 2008

(51) Int. Cl. A61M 16/00

(52) U.S. Cl. 128/204.22; 128/204.23

(57) ABSTRACT

A medical diagnostic tool provides a fixture mounted on a cart, the fixture having an inspiratory conduit joined with an expiratory conduit, with both joined to a patient interface. An air inlet, and an inlet valve are interconnected in series by the inspiratory conduit; and a manometer is interconnected with the inspiratory conduit between the inlet valve and the patient interface. An air outlet, a spirometer, and an outlet valve are interconnected in series by the expiratory conduit, and a side-stream capnometer is interconnected with the expiratory conduit between the outlet valve and the patient interface. The inlet valve and the outlet valve each enable air flow in only one direction. The inlet valve further provides a shutoff, which when activated, prevents air flow through the inlet valve.

### BACKGROUND OF THE INVENTION

1. Field of the Present Disclosure

This disclosure relates generally to medical monitoring and recording apparatus in the field of respiratory ailment and particularly to a method of determining respiratory status of those breathing on a ventilator.

2. Description of Related Art Including Information Disclosed Under 37 CFR 1.97 and 1.98

Be'eri, U.S. 2007/0199566, discloses exemplary embodiments that provide a respiratory device that can perform mechanical ventilation and/or inexsufflation. The respiratory device can include a mechanical medical ventilator, a sensor, a display and a processor. The mechanical medical ventilator assists a patient with the respiratory cycle. The sensor can measure an intra-thoracic respiratory parameter during the respiratory cycle. The display can display a graphical representation that dynamically depicts at least one of a patient's lung or thorax based on the intra-thoracic respiratory parameter in real-time during the respiratory cycle. The processor can update the graphical representation on the display in real-time based on the respiratory parameter. The processor updates the graphical representation to depict at least one of an expansion or a contraction of at least one of the lung or thorax during the respiratory cycle.

Merilainen, U.S. Pat. No. 4,856,531, discloses a device intended for monitoring the carbon dioxide output, oxygen consumption and respiration quotient of a patient connected to a respirator. The device comprises O.sub.2 and CO.sub.2 analyzers, a mixing chamber, a constant flow fan, a gas collector hose and magnetic valves. The carbon dioxide output and oxygen consumption are directly calculated from the carbon dioxide content of gas mixed with constant air flow from said mixing chamber, from the carbon dioxide and oxygen contents of the gas in said mixing chamber, and from the oxygen content of the gas delivered into a patient by said respirator.

Maher, U.S. Pat. No. 5,103,814, discloses a ventilator that has automatic controls with a particular control sequence to progressively wean the patient from mechanical ventilation, but that detects and maintains the patient's condition against the patient's inability to resume normal respiration. The ventilator non-invasively monitors body oxygen saturation level to insure adequate respiration, with minimum excess oxygen exposure, and monitors exhaled tidal carbon dioxide levels to control mechanically assisted respiration rate.

Cewers, U.S. Pat. No. 5,816,242, discloses a device for transmitting information via a patient tube from a location near the patient to an intensive care or anesthetic machine. At least one signal source is arranged at one end portion of the tube to deliver information-carrying signals which propagate longitudinally through the medium inside the tube. At least one receiver is arranged at the other end of the tube to receive the signals.

Hecker et al., U.S. Pat. No. 5,957,128, discloses a method and a device for determination of functional residual capacity (FRC) by introduction of helium or another inert gas mixture. According to the invention, a measurement apparatus measures the density of the gas mixture upon inspiration and upon expiration at the mouthpiece of a tube or at a mask during forced ventilation of a patient over a plurality of respiratory cycles. The FRC is determined from the difference in the gas concentrations.

Heinonen, U.S. Pat. No. 6,139,506, discloses a method for determining pulmonary functional residual capacity (FRC). A given amount of indicator gas is delivered into the breathing gases flowing into the lungs of a subject in a selected number of sequential breaths. The amounts of indicator gas delivered during the selected number of breaths are summed to provide a cumulative total. The amount of indicator gas exhaled in the number of sequential breaths is summed to provide a cumulative total. An indication of the concentration of indicator gas in the lungs of the subject is obtained for said two or more breaths. Using the calculated quantities as measured variables, at least two measured value data sets are formed in which the product of the indicator gas concentration and a regression coefficient comprising the functional residual capacity plus the product of the cumulative total of exhaled indicator gas and a regression coefficient K equals the cumulative total of the delivered indicator gas. Multi dimensional regression analysis is carried out using the data sets to obtain values for K and FRC fitted to said data sets. The value FRC so obtained is a determination of function residual capacity.

Gedeon, U.S. Pat. No. 6,302,851, discloses a method and apparatus for determining a pulmonary function parameter, EVG, indicative of a living subject's effective lung volume, namely the lung volume in which gas exchange between respiratory air and pulmonary blood takes place efficiently. The apparatus carries out the steps of the method: (1) determining for a first breath during normal steady state breathing of the subject the end-tidal carbon dioxide or oxygen concentration P.sub.et1 and the average rate of flow V.sub.a1, over the duration T.sub.1 of the breath, of expired carbon dioxide or oxygen, determining for a second breath comprising a breath-hold period the end-tidal carbon dioxide or oxygen concentration P.sub.et2 and the average rate of flow V.sub.a2, over the duration T.sub.2 of the breath, of expired carbon dioxide or oxygen, and determining EVG as a quantity proportional to the ratio of the difference between said average flow rates V.sub.a1 and V.sub.a2 to the difference between said end-tidal concentrations P.sub.et2 and P.sub.et1.

Koch et al., U.S. Pat. No. 6,544,191, discloses a process for determining the functional residual capacity (FRC) of the lungs during respiration. An environmentally friendly trace gas is used in a process and system for determining the FRC by using fluoropropanes as a trace gas. Values for the FRC can thus be calculated, resolved for individual breaths, from the expiratory trace gas concentration and the expired breathing gas volume and they can be used for determining the FRC depending on their convergence behavior.

Jonson, U.S. Pat. No. 6,709,405, discloses an apparatus and method for examining the pulmonary mechanics of a respiratory system, in order to obtain information about the mechanical properties of the respiratory system's lungs, during an expiration of a flow of gas streaming out of the respiratory system is modulated, the volume of gas streaming out of the respiratory system is determined, the variation in pressure in the respiratory system is determined, and an expiratory pressure-volume relationship is determined from the expiratory volume and the expiratory variation in pressure.

The related art described above discloses apparatus and methods for determining medical status of the human respiratory function. However, the prior art fails to disclose a movable cart having an optimized apparatus for testing respiration rate, pulse rate, pulse-ox, ETCO₂, patient suction capacity and lung capacity. The present disclosure distinguishes over the prior art providing heretofore unknown advantages as described in the following summary.

### BRIEF SUMMARY OF THE INVENTION

This disclosure teaches certain benefits in construction and use which give rise to the objectives described below. The present apparatus is a medical diagnostic tool providing a fixture mounted on a cart, the fixture having an inspiratory conduit joined with an expiratory conduit, with both joined to a patient interface. An air inlet, and an inlet valve are interconnected in series by the inspiratory conduit; and a manometer is interconnected with the inspiratory conduit between the inlet valve and the patient interface. An air outlet, a spirometer, and an outlet valve are interconnected in series by the expiratory conduit, and a side-stream capnometer is interconnected with the expiratory conduit between the outlet valve and the patient interface. The inlet valve and the outlet valve each enable air flow in only one direction with the inlet valve oriented in the inspiratory conduit for allowing air flow to only pass to the patient interface, but not in the opposite direction, while the output valve is oriented in the expiratory conduit for allowing air flow to only pass to the air outlet, but not in the opposite direction. The inlet valve further provides a shutoff, which when activated, prevents all air flow through the inlet valve.

A primary objective inherent in the above described apparatus and method of use is to provide advantages not taught by the prior art.

Another objective is to provide an integrated mobile apparatus having an equipment assembly capable of fast, accurate and repeatable measurement of patient lung capacity and health.

A further objective is to provide such an apparatus that is optimized for quick application when needed.

A still further objective is to provide such an apparatus that is able to measure patient suction capacity as well as inspiratory and expiratory lung capacity.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

An embodiment of the present invention is illustrated in the accompanying drawings. In such drawings:

FIG. 1 is an elevational view of the present invention;

FIG. 2 is an elemental schematic diagram thereof; and

FIG. 3 is a typical readout or printout of a spirometer thereof.

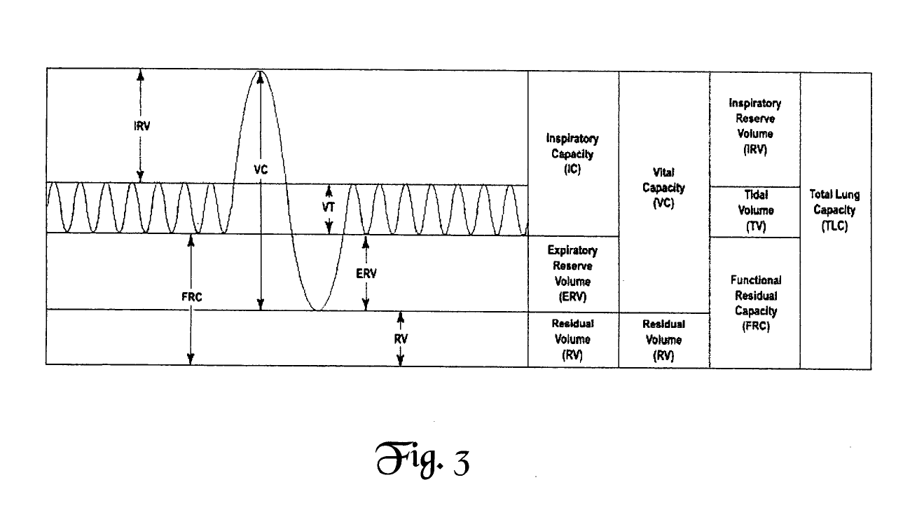

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications to what is described herein without departing from its spirit and scope. Therefore, it should be understood that what is illustrated is set forth only for the purposes of example and should not be taken as a limitation on the scope of the present apparatus and its method of use.

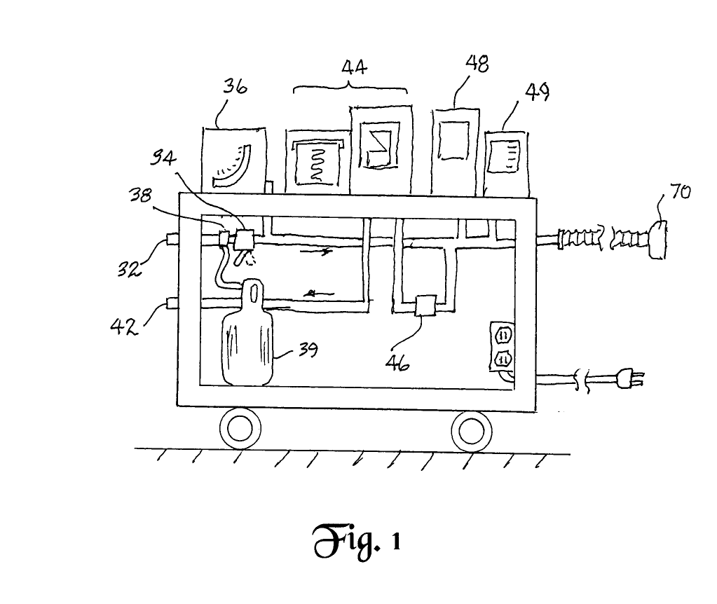

The present invention is described now in detail as a medical diagnostic cart apparatus and method of use. As shown in the elemental schematic of FIG. 1, a fixture 10 is mounted on a cart 20 of the type that may be wheeled from place to place, and in the preferred application, from patient bed to patient bed in a hospital or other medical setting. Fixture 10 has an inspiratory conduit 30, as shown in FIG. 1. Conduit 30 is joined with an expiratory conduit 40 at a tee connector 50. Tee connector 50 is further joined with a common conduit 60 that terminates at a patient interface 70, typically an endotracheal tube, a simple mouthpiece or a tracheostomy tube. A patient is therefore able to breathe normally, inhaling through the inspiratory conduit 30 and exhaling through the expiratory conduit 40.

An air inlet 32, and an inlet valve 34 are interconnected in series by the inspiratory conduit 30, and a manometer 36 is further interconnected with the inspiratory conduit 30 at a location between the inlet valve 34 and the tee connector 50. This location is critical because it is necessary to maximize conduit conduction for accurate manometer readings.

An air outlet 42, a spirometer 44, and an outlet valve 46 are interconnected in series by the expiratory conduit 40; and a side-stream capnometer 48 is further interconnected with the expiratory conduit 40 between the outlet valve 46 and the tee connector 50. This location is critical because it is necessary to maximize conduit conduction for accurate capnometer readings. Capnography is the monitoring of the concentration or partial pressure of carbon dioxide (CO₂) in the respiratory gases. Its main use has been as a monitoring tool for use during anesthesia and intensive care. It is usually presented as a graph of expiratory CO₂ plotted against time, or, less commonly, but more usefully, expired volume. The plot may also show the inspired CO₂, which is of interest when rebreathing systems are being used. The capnogram is a direct monitor of the inhaled and exhaled concentration or partial pressure of CO₂, and an indirect monitor of the CO₂ partial pressure in the arterial blood. In healthy individuals, the difference between arterial blood and expired gas CO₂ partial pressures is small. In the presence of most forms of lung disease, and some forms of congenital heart disease (the cyanotic lesions) the difference between arterial blood and expired gas increases and can exceed 1 kPa. Capnometer 48 also uses a pulse oximeter 49, a particularly convenient non-invasive measurement instrument. Typically it has a pair of small light-emitting diodes (LED's) facing a photodiode through a translucent part of the patient's body, usually a fingertip or an earlobe. One LED is red, with wavelength of 660 nm, and the other is infrared, 905, 910, or 940 nm. Absorption at these wavelengths differs significantly between oxyhemoglobin and its deoxygenated form, therefore from the ratio of the absorption of the red and infrared light the oxy/deoxyhemoglobin ratio can be calculated. The monitored signal bounces in time with the heart beat because the arterial blood vessels expand and contract with each heartbeat. By examining only the varying part of the absorption spectrum, a monitor can discern only the absorption caused by arterial blood. Thus, detecting a pulse is essential to the operation of a pulse oximeter and it will not function if there is none.

The spirometer 44 is a well known apparatus for measuring the volume of air inspired and expired by the lungs. It is a precision differential pressure transducer for the measurements of respiration flow rates. The spirometer 44 records the amount of air and the rate of air that is breathed in and out over a specified time. The spirometer 44 and attached flow head function together as a pneumotachometer, with an output signal proportional to airflow. The output produced by a spirometer 44 is called a kymograph trace; See FIG. 4 which illustrates a prototypical output of a ‘spirometer’. The vertical axis signifies the volume and the horizontal axis signifies time. The bottom left corner equals zero lung volume at the start of the spirometer recording session. The first small amplitude part of the sinusoid depicts repeated resting state involuntary breathing. The amplitude of this small sinusoid corresponds to the ‘Tidal Volume’. The large positive amplitude spike represents voluntary inspiration to maximal volume or ‘Total Lung Volume’. The large negative amplitude spike represents forced expiration to the lowest possible physiological lung volume called ‘Residual Volume’. From this, vital capacity, tidal volume, breathing rate and ventilation rate (tidal volume x breathing rate) can be calculated. From the overall decline on the graph, the oxygen uptake can also be measured. Vital capacity is the maximum amount of air a person can expel from the lungs after first filling the lungs to their maximum extent and then expiring to the maximum extent (about 4600 milliliters). It equals the inspiratory reserve volume plus the tidal volume plus the expiratory reserve volume.

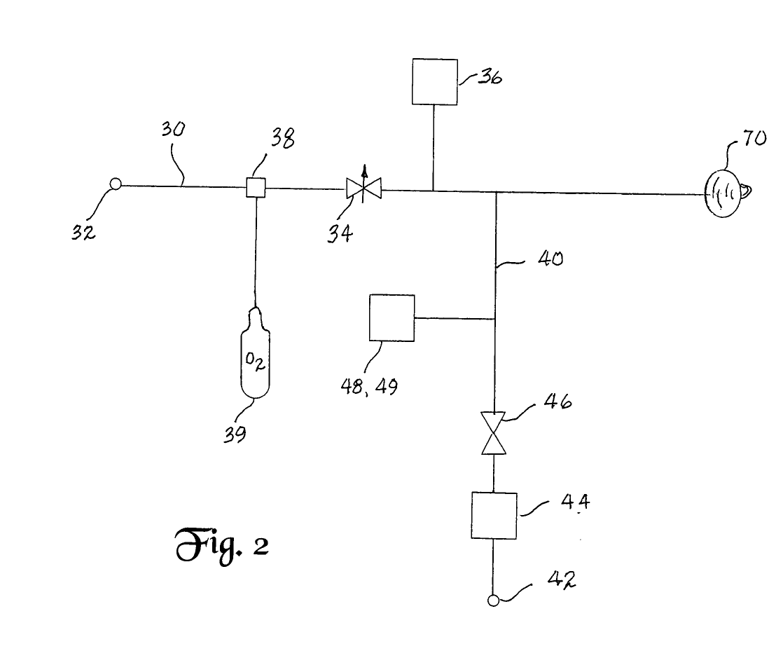

The inspiratory and expiratory conduits, respectively elements 30 and 40, may alternately terminate directly at the patient interface 70, a mouthpiece for instance, in order to eliminate conductance loss through the common conduit 60 and tee connector 50. This enablement is shown schematically in FIG. 2.

Preferably, cart 20 provides an electrical power interface 80 such as an electrical extension cord with AC receptacles permanently attached to cart 20. This provides operating current to the manometer 36, the spirometer 44 and the capnometer 48, where such current is required, that is, when such components are not self powered or do not require electrical power.

Each of inlet valve 34 and outlet valve 46 are of the type that allow air flow in only one direction while preventing air flow in the reverse direction. The inlet valve 34 is mounted with orientation in the inspiratory conduit 30 for allowing air flow to only pass to the patient interface 70 while the output valve is oriented in the expiratory conduit 40 so as to allow air flow to only pass to the air outlet 42. The inlet valve 34 further has a shutoff feature, which, when activated, prevents any air flow through inlet valve 34. The valves 34 and 46 and their respective capabilities and features are not inventive but are well known being “off-the-shelf” components.

Preferably, the above described medical diagnostic cart and apparatus further has an inspirator, a device such as a venturi tube or an orifice plate, which mixes oxygen with inhalation of atmospheric air in a precise ratio. The pressure of the air flow is used to draw in and mix the oxygen. Venturi tube 38 is mounted in the inspiratory conduit 30 in a position between the air inlet 32 and the inlet valve 34. In this embodiment a source of oxygen 39 may be interconnected with the venturi tube 38 to allow oxygen to be drawn into the inspiratory conduit 30 as a patient, who requires supplemental oxygen, draws air in. Venturi tube 38 may be adjusted for percent oxygen admitted into the inflowing air stream. Preferably, the cart 20 is facilitated for carrying a bottle of compressed oxygen gas and also an oxygen conduit for connecting the oxygen bottle with the venturi tube 38. Where available, the oxygen conduit may be used to connect a hospital oxygen wall outlet directly to the venture tube 38, bypassing the use of a portable oxygen bottle.

The above described apparatus is used for monitoring patients 5 for diagnostic purposes and in particular, to determine when a patient 5 who is on a ventilator may be weaned from it.

In use, the cart 20 is moved to the patent's bedside and the patient interface 70 is connected to the patient 5. First, tidal volume of each single patient breath is recorded when breathing normally, and an average tidal volume for a single patient breath when breathing normally over a one-minute period is automatically calculated using the spirometer 44. At this time the inlet shutoff valve is open. At the same time, the capnometer measures respiration rate, pulse rate and end tidal CO₂ level.

Next, with the inlet shutoff valve 34 closed, a maximum patient suction is measured and recorded using the manometer. Preferably, a technician has control over the shutoff function of valve 34 and holds the valve 34 open so that the patient can draw air in, breathing normally. Then requesting that the patient exhale fully, valve 34 is closed and the patient is asked to inhale with maximum force while the manometer 36 is monitored. Immediately thereafter, valve 34 is opened again so that the patient is able to breathe freely once again.

Finally, vital capacity is measured using the spirometer by having the patient take a maximum deep breath and then exhaling it.

Further details relating to the construction and deployment of a respiratory apparatus are found in U.S. application publication 2007/0199566, the relevant disclosure of which is included by reference thereto as if fully set forth herein.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. A medical diagnostic cart apparatus comprising:
a fixture mounted on a mobile cart, the fixture having an inspiratory conduit joined with an expiratory conduit, the conduits further joined with a patient interface;
an air inlet, and an inlet valve interconnected in series by the inspiratory conduit; and a manometer interconnected with the inspiratory conduit between the inlet valve and the patient interface;
an air outlet, a spirometer, and an outlet valve interconnected in series by the expiratory conduit; and a capnometer interconnected with the expiratory conduit between the outlet valve and the patient interface;
the inlet valve and the outlet valve each having construction enabling air flow in only one direction therethrough, the inlet valve oriented in the inspiratory conduit for enabling air flow to only pass toward the patient interface, the output valve oriented in the expiratory conduit for enabling air flow to only pass toward the air outlet; the inlet valve further having a shutoff, the shutoff, when actuated, preventing air flow through the inlet valve.

2. The medical diagnostic cart apparatus of claim 1 further comprising an electrical power interface providing operating current.

3. The medical diagnostic cart apparatus of claim 1 further comprising an inspirator positioned between the air inlet and the inlet valve in the inspiratory conduit.

4. The medical diagnostic cart apparatus of claim 3 further comprising a source of oxygen interconnected with the inspirator.

5. The medical diagnostic cart apparatus of claim 3 further comprising an oxygen conduit interconnected with the inspirator.

6. A medical diagnostic apparatus comprising:
a fixture having an inspiratory conduit joined with an expiratory conduit, the conduits further joined with a patient interface;
an air inlet, and an inlet valve interconnected in series by the inspiratory conduit; and a manometer interconnected with the inspiratory conduit between the inlet valve and the patient interface;
an air outlet, a spirometer, and an outlet valve interconnected in series by the expiratory conduit; and a capnometer interconnected with the expiratory conduit between the outlet valve and the patient interface;
the inlet valve and the outlet valve each having construction enabling air flow in only one direction therethrough, the inlet valve oriented in the inspiratory conduit for enabling air flow to only pass toward the patient interface, the output valve oriented in the expiratory conduit for enabling air flow to only pass toward the air outlet; the inlet valve further having a shutoff, the shutoff, when actuated, preventing air flow through the inlet valve.

7. The medical diagnostic apparatus of claim 6 further comprising an inspirator positioned between the air inlet and the inlet valve in the inspiratory conduit.

8. The medical diagnostic apparatus of claim 7 further comprising a source of oxygen interconnected with the inspirator.

9. The medical diagnostic apparatus of claim 7 further comprising an oxygen conduit interconnected with the inspirator.

10. A medical diagnostic method comprising the steps of:
a) providing a fixture having an inspiratory conduit joined with an expiratory conduit, both further joined with a patient interface;
b) interconnecting an air inlet, and a one-way flow inlet shutoff valve in series in the inspiratory conduit; and connecting a manometer with the inspiratory conduit between the inlet valve and the patient interface;
c) interconnecting an air outlet, a spirometer, and a one-way flow outlet valve in series in the expiratory conduit; and connecting a capnometer with the expiratory conduit between the outlet valve and the patient interface;
d) interconnecting the patient interface with a patient;
e) recording tidal volume of each single patient breath when breathing normally, and average tidal volume for a single patient breath when breathing normally over a measured time period using the spirometer, with the inlet shutoff valve open;
f) recording respiration rate, pulse rate and end tidal CO₂ level using the capnometer, with the inlet shutoff valve open;
g) recording maximum patient suction using the manometer, with the inlet shutoff valve closed; and
h) recording vital capacity using the spirometer.

11. The medical diagnostic method of claim 10 comprising the further step of interconnecting an inspirator, positioned between the air inlet and the inlet valve, in the inspiratory conduit.

12. The medical diagnostic method of claim 11 comprising the further step of interconnecting a source of oxygen with the inspirator and adjusting a level of inspired oxygen.
